(19) United States
(12) Patent Application Publication
(10) Pub. No.: US 2016/0002596 A1
(43) Pub. Date: Jan. 7, 2016

(54) COMPOSITION FOR EX VIVO CULTURING OF A CORNEAL ENDOTHELIAL CELL AND A METHOD FOR USING THE COMPOSITION

(71) Applicant: National Taiwan University, Taipei (TW)

(72) Inventors: I-Jong Wang, Taipei City (TW); Wei-Ting Ho, New Taipei City (TW); Ting-Hsuan Chiang, Taipei City (TW); I-Tsen Lin, Taipei City (TW)

(21) Appl. No.: 14/791,401

(22) Filed: Jul. 3, 2015

(30) Foreign Application Priority Data
Jul. 4, 2014 (TW) 103123100

(51) Int. Cl. C12N 5/079

(52) U.S. Cl. CPC C12N 5/0621; C12N 2501/734

(57) ABSTRACT

A composition for ex vivo culture of corneal endothelial cell and a method for used the composition are disclosed. The composition or the method is applied to ex vitro culture of the corneal endothelial cell to maintain the corneal endothelial cell in a hexagonal phenotype, and to prevent the corneal endothelial cell from endothelial-mesenchymal transformation (EnMT). Therefore, the corneal endothelial cell can maintain normal phenotype and function and can be used in the further application. The composition at least comprises an effective concentrated matrix metalloproteinase inhibitor (MMPI).

### FIELD OF THE INVENTION

Embodiments of the present invention relate to a composition for ex vivo culture of a corneal endothelial cell and a method for using the composition, especially to a composition used to maintain the corneal endothelial cell shape while ex vivo culturing and a method thereof.

### BACKGROUND

Human corneal endothelial cells form a monolayer in the posterior part of cornea, which act as a barrier between the anterior chamber and the corneal stroma, and have the ability to maintain corneal clarity by active pumping. Thus, corneal endothelial cells are important to maintain corneal transparency and the visual acuity.

The functional disorder or decline of the corneal endothelial cells due to different reasons, such as getting older age, wearing the contact lens for a long time, experiencing an intraocular surgery, or has congenital corneal endothelial cell deficiencies, will result in corneal edema, reduced transparency of cornea, and decreased visual acuity. However, corneal endothelial cells have limited regenerating capacity. Thus, patients with severe corneal endothelial decompensation will need corneal transplant surgery to restore vision.

The corneal transplantation is the most common therapy to treat severe corneal endothelial cells disorder. However, the surgery is restricted by the donor source and the selected condition, and has risk of allograft rejection. Therefore, many researchers try hard to develop a method for ex vivo culturing of corneal endothelial cells to solve such problems.

There are two barriers exist in the ex vivo corneal endothelial cell culture, including stimulating the proliferation and maintenance of the phenotype of the corneal endothelial cell to have the normal function.

### SOME EXEMPLARY EMBODIMENTS

These and other needs are addressed by the present invention, wherein an approach is provided for a composition for ex vivo culturing of a corneal endothelial cell and a method for using the composition.

One aspect of the present invention is to provide a growth regulator for ex vivo culturing of a corneal endothelial cell added in a corneal endothelial cell culture medium to maintain the corneal endothelial cell in a hexagonal phenotype and prevent the corneal endothelial cell from endothelial-mesenchymal transition and lose its function. In an embodiment, the growth regulator at least comprises an effective concentrated matrix metalloproteinase inhibitor (MMPI).

Another aspect of the present invention is to provide an inhibitor for preventing an ex vivo cultured corneal endothelial cell from endothelial-mesenchymal transition.

Another yet aspect of the present invention is to provide a method to prevent a corneal endothelial cell from endothelial-mesenchymal transition, comprising steps of obtaining the corneal endothelial cell from a donor, incubating the corneal endothelial cell in a corneal endothelial cell culture medium, and adding an effective concentrated matrix metalloproteinase inhibitor in a specific time point. The specific time point is in the initial or middle of the incubation. The concentration of the MMPI is at least 1 μM.

### BRIEF DESCRIPTION OF THE DRAWINGS

The invention is illustrated by way of example, and not by way of limitation, in the figures of the accompanying drawings in which like reference numerals refer to similar elements and in which:

FIG. 1 is a photograph showing the shape and immuno-fluorescence staining by different antibodies of ex vivo culturing of the corneal endothelial cell.

FIG. 2 is a photograph showing immuno-fluorescence staining of ex vivo culturing of corneal endothelial cell by different antibodies.

FIG. 3 is a graph showing the corneal endothelial cell growth under different incubation condition.

FIG. 4 is a photograph showing the influence of Batimastat or GM6001 in ex vivo corneal endothelial cell culture.

FIG. 5 is a western blot assay photograph showing the expression of active beta-catenin, an indicator of endothelial-mesenchymal transition, by the corneal endothelial cell treated with different concentrations of Marimastat or ADAM10 specific inhibitor GI254023X.

### DESCRIPTION OF THE PREFERRED EMBODIMENT

A composition for ex vivo culture of a corneal endothelial cell and a method for using the composition are disclosed. In the following description, for the purposes of explanation, numerous specific details are set forth in order to provide a thorough understanding of the present invention. It is apparent, however, to one skilled in the art that the invention may be practiced without specific details or with an equivalent arrangement. In other instances, well-known structures and devices are shown in block diagram form in order to avoid unnecessarily obscuring the embodiments of the present invention.

All disclosed embodiments or experiences follow the “statement for the use of animal in ophthalmic and vision research” of Association for research in vision and ophthalmic (ARVO) and are approved by the Institutional animal care and use committee of National Taiwan University Hospital.

Corneal endothelial cells are collected from bovine eye. The bovine eye is immersed in the iodine for 3 minutes for sterilization, and washed by the phosphate buffer saline solution (PBS). The corneal button is procured, and the Descemet's membrane is peeled from the corneal button, reacted with the Trypsin at 37° C. for 30 minutes, and centrifuged to collect the corneal endothelial cells.

The medium used in the embodiments is supplemented hormonal epithelial medium (SHEM), that is prepared by same volumes of Dulbecco' s modified eagle medium (DEME) and Ham's nutrient mixture F12 medium (Ham 12) added with 5% bovine serum (FBS), 0.5% dimethyl sulfoxide (DMSO), 2 ng/ml human epidermal growth factor(hEGF), 5 ug/ml insulin, 5 ug/ml transferring, 5 ng/ml selenium, 1 nM Cholera toxin (CT), 50 ug/m1 gentamicin and 1.25 ug/ml amphotericin B.

The corneal endothelial cells are incubated at 37° C. in the 5% carbon dioxide incubator.

### Example 1: Immunohistochemistry (IHC) Assay

The corneal endothelial cells is fixed on the glass slide with 4% paraformaldehyde at room temperature for 30 minutes, permeabilized with 0.5% triton X-100 for 5 minutes and reacted with 10% bovine serum albumin (BSA) for 5 minutes to obtain a corneal endothelial cell biopsy.

The corneal endothelial cells are immune-stained with different first antibodies (beta-catenin, active beta-catenin (ABC), snail or slug) at 4° C. overnight. Snail and slug are regulators related to the endothelial-mesenchymal transition (EnMT) process.

The corneal endothelial cells are washed with the PBS for 15 minutes twice, then reacts with Alexa-Fluro® 568 conjugated-linked second antibody (1:100) at room temperature for 1 hour and counterstained with 4′,6-diamidino-2-phenylindole (DAPI) (1:5000) at room temperature for 5 minutes. The specimen is washed and mounted in the fluorescence mounting solution (VectA Mount, Vector Laboratories, Bulingame, Calif.) and is observed under the confocal spectral microscope (Leica, TCS SP5).

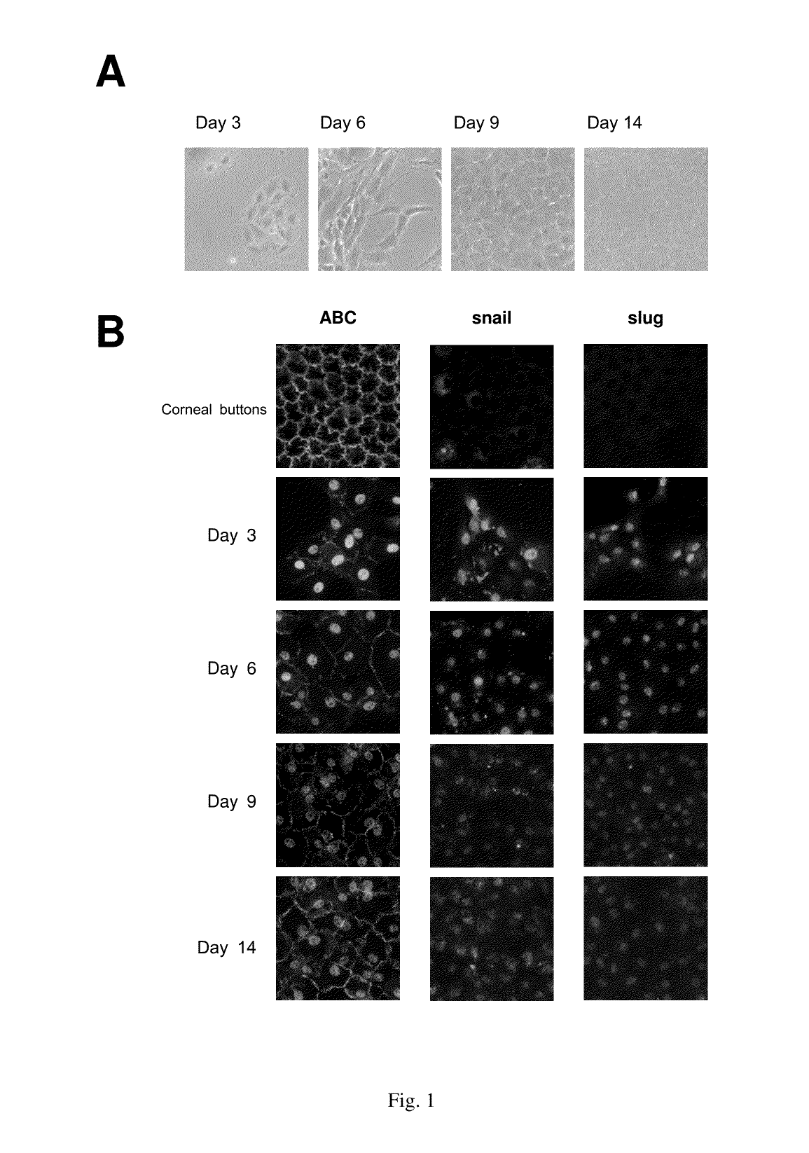

With reference to FIG. 1, FIG. 1 is a photograph showing the shape and immuno-fluorescence staining result of ex vivo culturing of the corneal endothelial cell. The corneal endothelial cells undergo endothelial-mesenchymal transition during ex vivo culture, which is evidenced by nuclear translocation of ABC, snail, and slug.

### Example 2: The Effect of Matrix Metalloproteinase Inhibitor (MMPI) on the Phenotypic Change of Ex Vivo Cultured Corneal Endothelial Cells

In this example, the selected MMPI is Marimastat. However, any person with ordinary skill will understand that Marimastat can be replaced by any other MMPI. The cultured corneal endothelial cells were divided into 6 groups and were harvested on day 3 and day 9. The culture condition was described as follows:
Group 1: the corneal endothelial cells were cultured in SHEM for 9 days;
Group 2: the corneal endothelial cells were cultured in SHEM added with 10 uM MMPI for 9 days;
Group 3: the corneal endothelial cells were cultured in SHEM added with 10 uM Y-27632, a ROCK inhibitor used to stimulate the proliferation of corneal endothelial cells, for 9 days;
Group 4: the corneal endothelial cells were incubated in SHEM added with 10 uM Y-27632 and 10 uM MMPI simultaneously for 9 days;
Group 5: the corneal endothelial cells were cultured in SHEM added with 10 uM Y-27632 for the first 3 days, and in SHEM only for the next 6 days; and
Group 6: the corneal endothelial cells were cultured in SHEM added with 10 uM Y-27632 for the first 3 days, and in SHEM with 10 uM MMPI for the next 6 days.

With reference to FIG. 2, FIG. 2 is the immunofluorescence staining of cells on day 3 and day 9 using the antibody against active beta-catenin, an indicator of EnMT. Corneal endothelial cells of group 3 or group 5 exhibited enhanced proliferation in the presence of Y-27632. However, the cell shape became elongated and fibroblast-like instead of hexagonal. This result indicated that Y-27632 was able to promote corneal endothelial cell growth, but the cell morphology and thus function may be jeopardized.

The corneal endothelial cells in group 2 or group 4 cultured in the presence of MMPI maintained hexagonal morphology after 9 days. Adding MMPI during cell culture helped to maintain the phenotype of ex vivo cultured corneal endothelial cells.

Further, the morphology of corneal endothelial cells in group 6 can also be reversed to hexagonal shape after cultured in SHEM with MMPI for the last 6 days, despite Y-27632 in the first 3 days (referred to the phenotype of day 3 and day 6 of group 6).

### Example 3: The Effect of MMPI on the Growth of Ex Vivo Cultured Corneal Endothelial Cell

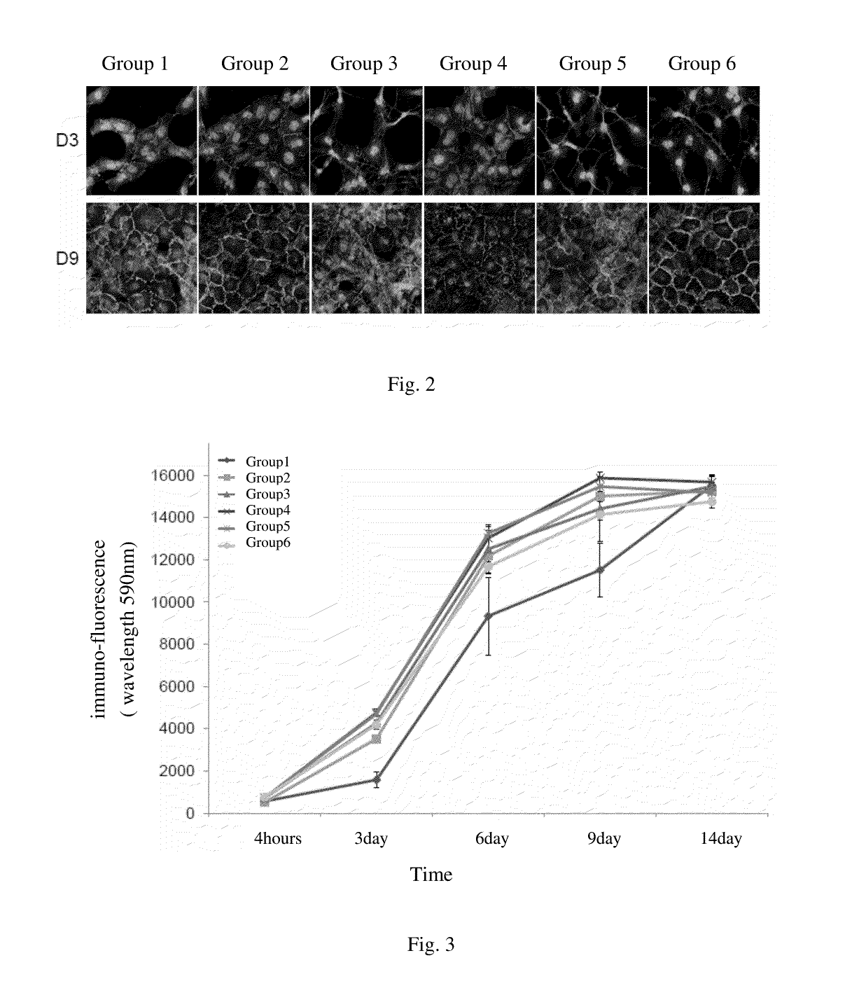

With reference to FIG. 3, FIG. 3 demonstrated the proliferation of corneal endothelial cells under different culture condition. Y-27632 is a known growth stimulator for corneal endothelial cells that is able to increase the ex vivo cell proliferation. The cell growth in the group 2, 4 and 6 (MMPI added in the SHEM) was not obviously inhibited or reduced, suggesting that MMPI has the potential to be added in the medium to preserve the cell phenotype.

### Exmaple 4: Different MMPIs Effect the Phenotype of Ex Vivo Cultured Corneal Endothelial Cell

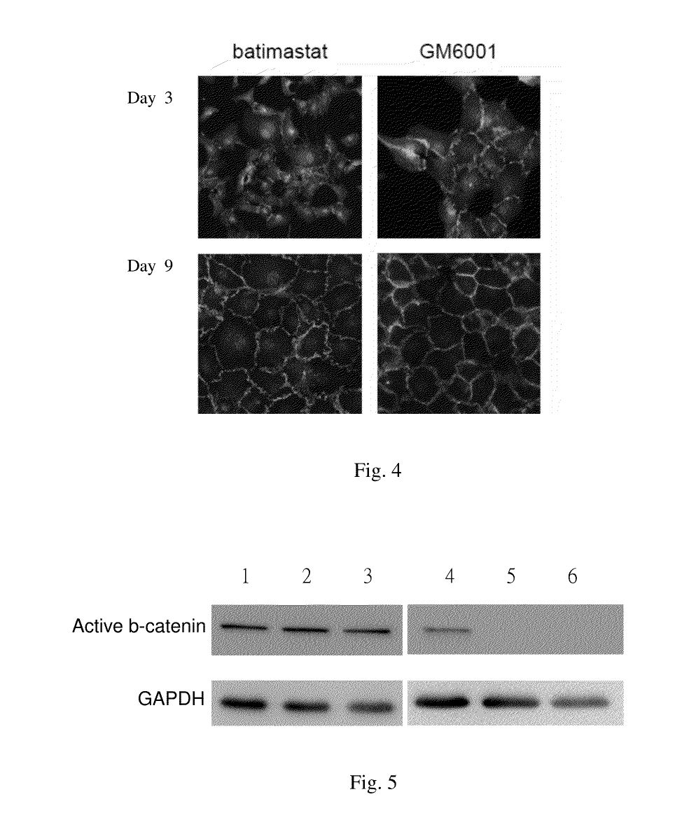

This example is similar to example 2, with Batimastat or GM6001 added in the medium instead of Marimastat. Batimastat and GM6001 are broad-spectrum MMPI similar to Marimastat. With reference to FIG. 4, FIG. 4 was the immunofluorescence staining using the antibody against active beta-catenin, which showed that after adding into the culture medium, Batimastat or GM6001 was capable to maintain the corneal endothelial cell shaped as Marimastat.

With reference to FIG. 5, FIG. 5 was the Western blot analysis showing the expression level of active beta-catenin (ABC) in corneal endothelial cells treated with different concentrations of Marimastat or ADAM10 (ADAM metallopeptidase domain 10) specific inhibitor GI254023X. In group 1-3, the corneal endothelial cells were incubated in the SHEM added with 100 nM, 1 uM or 10 uM of GI254023X, respectively. In group 4-6, cells were incubated in the SHEM added with 100 nM, 1 uM or 10 uM of Marimastat, respectively. In group 1-3, there was no reduction in the expression level of ABC in the presence of ADAM10 specific inhibitor, GI254023X. In group 4, ABC expression level was high despite 100 nM of Marimastat. However, the expression of ABC became significantly reduced with Marimastat more than 1 uM.

Accordingly, the MMPI is able to be applied as a growth regulator at the beginning or during the culture of corneal endothelial cells to maintain the cell shape and to prevent the cells from EnMT. This benefit has the potential to be further employed in the regenerative medicine to improve the quality of ex vivo cultured corneal endothelial cells.

While the invention has been described in connection with a number of embodiments and implementations, the invention is not so limited but covers various obvious modifications and equivalent arrangements, which fall within the purview of the appended claims. Although features of the invention are expressed in certain combinations among the claims, it is contemplated that these features can be arranged in any combination and order.

What is claimed is:

1. A growth regulator for ex vivo culturing of a corneal endothelial cell, at least comprises an effective concentrated matrix metalloproteinase inhibitor (MMPI). The regulator is added in the culture medium to maintain the phenotype and function of corneal endothelial cells and prevent the cells from endothelial-mesenchymal transformating and loss of function.

2. The growth regulator as claimed in claim 1, wherein the concentration of the MMPI is at least 1 uM.

3. An inhibitor for preventing an ex vivo cultured corneal endothelial cell form endothelial-mesenchymal transformation, at least comprises an effective matrix metalloproteinase inhibitor (MMPI). The inhibitor is added in the culture medium to maintain the phenotype and function of corneal endothelial cells and prevent the cells from endothelial-mesenchymal transformating and loss of function.

4. The growth regulator as claimed in claim 3, wherein the concentration of the MMPI is at least 1 uM.

5. A method to prevent a corneal endothelial cells from endothelial-mesenchymal transformation comprising:
obtaining the corneal endothelial cell from a donor;
incubating the corneal endothelial cell in a corneal endothelial cell culture medium; and
adding an effective concentration of matrix metalloproteinase inhibitor in a specific time point.

6. The method as claimed in claim 5, wherein the concentration of the MMPI is at least 1 uM.

7. The method is as claimed in claim 5, wherein the specific time point is at the beginning or during the cell culture.

8. A method to maintain the phenotype of an ex vivo cultured corneal endothelial cells, comprising:
obtaining the corneal endothelial cell from a donor;
incubating the corneal endothelial cell in a corneal endothelial cell culture medium; and
adding an effective concentrated matrix metalloproteinase inhibitor in a specific time point.

9. The method as claimed in claim 8, wherein the concentration of the MMPI is at least 1 uM.

10. The method as claimed in claim 8, wherein the specific time point is at the beginning or during the cell culture.
